(19) United States
(12) Patent Application Publication
(10) Pub. No.: US 2005/0222270 A1
(43) Pub. Date: Oct. 6, 2005

(54) Prolonged administration of NMDA antagonist drug and safener drug to create improved stable neural homeostasis

(76) Inventors: John W. Olney, Ladue, MO (US); Ronald E. Harbut, Hummelstown, PA (US); Graeme E. Correll, Mackay (AU); Patrick D. Kelly, St. Louis, MO (US)

Correspondence Address: Patrick D. Kelly, 11939 Manchester #403, St. Louis, MO, 63131, US

(21) Appl. No.: 10/786,212

(22) Filed: Feb. 26, 2004

(51) Int. Cl. A61K 31/137

(52) U.S. Cl. 514/650

(57) ABSTRACT

An NMDA antagonist (such as ketamine) is administered with a safener (such as clonidine) in patients suffering from neurologic disorders other than pain. The ketamine is adminsitered at a dosage that causes slurred speech, for a span of several days. This treatment enables a patient's nervous system to return to a healthy “set point”, also called an improved stable neural homeostasis, in a manner similar to a broken bone healing itself if protected from jostling and reinjury by a cast. In at least some patients, this treatment can ease problems such as addictions to illegal or pain-killing drugs, nicotine, or alcohol, compulsive or criminal behavioral problems, severe depression, obsessive-compulsive disorders, phobias, etc. It may also provide some relief in some patients for problems such as chronic fatigue, chemical sensitivities, allergies, autoimmune disorders, and diabetes.

### RELATED APPLICATION

This application claims the benefit, under 35 USC 119(e), of provisional application 60/449,643, filed on Feb. 23, 2003.

### FIELD OF THE INVENTION

This invention is in the field of pharmacology, and more particularly relates to the use of a drug such as ketamine, which is normally used as a short-acting surgical anesthetic, in a different type of treatment to provide a stable and long-lasting improvement in a patient suffering from a problem having a major neurological component, such as tobacco or drug addiction, alcoholism, clinical depression or bipolar disorder, obsessive-compulsive disorder, etc.

### BACKGROUND OF THE INVENTION

Ketamine is the common name for a drug that is widely used as a surgical anesthetic. Two of its more important traits are: (i) it is cleared from circulating blood fairly rapidly, which enables anesthesiologists to bring an unconscious patient out of anesthesia more rapidly than can be achieved by using other types of anesthetics with longer durations of action; and, (ii) it is an NMDA antagonist drug, which means that it exerts anesthetic effects by suppressing and reducing activity at the so-called NMDA class of neuronal receptors. The term “NMDA antagonist” is used interchangeably herein with terms such as “NMDA receptor blocker” or “NMDA blocker drug”.

Because of their central role in this invention, and in the nervous systems of mammals and other vertebrates, NMDA receptors need to be understood. The following is an overview, for those who are not familiar with these receptors (or other glutamate receptors), or with NMDA blocker drugs. More extensive information is readily available in the scientific and medical literature.

NMDA receptors are the single most important type of neuronal receptor in the entire mammalian nervous system. They are the most common and widely distributed subclass of a larger class of receptors known as glutamate receptors (or, alternately, as excitatory amino acid receptors).

“Glutamate” refers to the ionized form of glutamic acid, an important amino acid. The ionized form and the “free acid” form of this amino acid are both present, in equilibrium, in any aqueous fluid. In biological fluids that are not highly acidic, the ionized form (glutamate) is present at much higher concentrations than the acid form (glutamic acid). However, the ionized and non-ionized forms are simply two versions of the same compound, which coexist in equilibrium in all cells and cellular fluids.

In animals, glutamate has two entirely different functions. First, it is one of the twenty “primary” amino acids that are used as the building blocks to create all protein, in all living organisms on earth. As such, glutamate is ubiquitous in nature, and it is found in all living cells.

Second: in vertebrate animals, glutamate also is a neurotransmitter. Glutamate molecules are used by neurons to transmit nerve impulses from one neuron to another, via a synapse (synaptic junction) between a transmitting neuron, and a receiving neuron.

In particular, glutamate is the single most important “excitatory” neurotransmitter in mammalian brains. To understand that statement, one needs to understand the crucial differences between “excitatory” neurotransmitters and “inhibitory” neurotransmitters.

There are roughly half a dozen different “inhibitory” neurotransmitters used by mammalian brains. These include dopamine, serotonin, gamma-amino-butyric acid (GABA), etc. These inhibitory transmitters act in a manner that is analogous to the tuner in a radio or television. In the same way that a tuner device in a radio or television set must choose from among numerous competing channels, and suppress any unwanted channels and signals so that a single coherent and desirable set of sounds and images can emerge, the inhibitory transmitters in a mammalian brain and spinal cord must also process an oversupply of candidate nerve signals, and filter out unwanted signals so that coherent perceptions, thoughts, and memories can emerge and rise above the chatter, noise, and distractions.

The “excitatory” neurotransmitters provide that oversupply of candidate signals. As mentioned above, glutamate is the most important excitatory neurotransmitter, in mammals (acetyl-choline is the second most important excitatory neurotransmitter; however, because glutamate is more difficult to explain or understand, many people who have heard of acetyl-choline have never heard of glutamate as a neurotransmitter). Therefore, glutamate (and glutamate receptors) are crucially important, throughout any mammalian central nervous system.

It should be noted, in passing, that glutamate receptors also can be activated by another primary amino acid, called aspartic acid (its ion is called aspartate). Therefore, glutamate receptors are sometimes referred to as “excitatory amino acid” (EAA) receptors. However, aspartate is used only rarely as a neurotransmitter, while glutamate is the truly dominant and major CNS neurotransmitter. Therefore, EAA receptors are referred to more commonly as glutamate receptors.

There are three subclasses of glutamate receptors inside the central nervous system of a mammal, and each subclass is named after a certain type of “probe drug” which selectively activates that particular subclass. NMDA receptors were named after a compound called N-methyl-D-aspartate (NMDA), because scientists learned in the 1980's that NMDA will strongly activate that one subclass of glutamate receptors, without trigger activity at the other subclasses of glutamate receptors. The NMDA compound does not occur in mammalian brains, and it is not used as an actual drug, for treating any medical conditions, since it will cause severe convulsions, due to its very potent activity in triggering NMDA receptors. However, since NMDA was the first known compound that could be used to identify and distinguish one particular subclass of glutamate receptors, those receptors were designated as NMDA receptors.

The other two subclasses of ionotropic glutamate receptors are called kainate receptors, and AMPA receptors, because of their reactions with two other probe drugs. They are much less widespread than NMDA receptors, and they are much more similar to each other than to NMDA receptors (i.e., both kainate and AMPA receptors can be triggered by a number of probe drugs that do not trigger NMDA receptors; this type of overlapping receptor activity is often referred to as “cross-affinity”). Therefore, kainate and AMPA receptors are often referred to collectively as “non-NMDA” receptors.

AMPA receptors formerly were called quisqualate (or QUIS) receptors, since they were first discovered to be activated by a probe drug called quisqualic acid. However, quisqualic acid also triggers a completely different class of metabotropic receptors. To avoid confusion, researchers today use a more selective probe drug, called alpha-amino-3-hydroxy-5-methylisoxazole-4-propionic acid (abbreviated as AMPA), as a probe drug for evaluating activity at AMPA receptors.

Glutamate Toxicity (Excitotoxicity)

Although glutamate plays an absolutely crucial and highly beneficial role as the main excitatory neurotransmitter inside the brain, its activity as a neurotransmitter also has a dangerous side, which can be explained as follows.

Under normal and healthy conditions, when a neuron is transmitting a nerve signal to another neuron, a molecule of glutamate is released by the transmitting neuron, at a synaptic junction between the two neurons. The glutamate molecule enters the fluid that fills the synaptic junction between the two neurons, and the glutamate briefly binds to the exposed portion of a receptor protein that is embedded in the cell membrane on the surface of the signal-receiving neuron. This binding reaction (between the glutamate and the receptor protein) leads to the opening of ion channels through the membrane that encloses the signal-receiving cell. This opening of ion channels in the membrane allows sodium ions (Na⁺) to flow into the neuron. The glutamate molecule is then released from the receptor protein, and it floats back into the fluid that fills the synaptic junction between the two neurons.

When a free glutamate molecule is released by a synaptic receptor protein, it is quickly grabbed by a transporter protein, as part of a series of reactions that cause the glutamate molecule to be pumped back into the interior of the neuron that originally released that glutamate molecule. This allows the signal-transmitting neuron to recycle and reuse that same glutamate molecule, in a later “firing” event.

The entire four-step process (glutamate release by a signal-transmitting neuron; binding of the glutamate to a receptor on the signal-receiving neuron; release of the glutamate by the receptor; and transport of the glutamate back into the signal-transmitting neuron) occurs within milliseconds. Although it is usually highly efficient, it is not perfect, and free glutamate molecules occasionally escape from synaptic junctions. When this occurs, the free glutamate molecules are usually absorbed by support cells called “glial cells”, which are present in brain and spinal tissue, but which cannot send or receive nerve signals.

Ketamine functions as a surgical anesthetic by blocking the activity of glutamate molecules at NMDA receptors. Because NMDA receptors are so common and widespread throughout the CNS of any human or other mammal, even a partial blockade of the NMDA receptor system, by a drug such as ketamine, can render a patient completely unconscious, during surgery.

In addition to that type of recognized medical use as a surgical anesthetic, it has been recognized for years that NMDA receptor blocker drugs also have a second potentially large and important medical use, because they may be able to help reduce and minimize brain damage after a stroke, cardiac arrest, near-suffocation, head or spinal injury, or other crisis that causes an interruption of blood flow or oxygen supply to the brain. Although this potential use for NMDA blocker drugs (which includes ketamine) is not directly related to the pain-control use disclosed herein, the reader should be familiar with that filed of research involving NMDA receptor blockers, to better understand this current invention.

To understand the potential use of NMDA blockers to reduce brain damage following a stroke or other crisis, one must focus on the glutamate transport system. As mentioned above, in a healthy brain, that transport system pumps free glutamate molecules back into the interiors of the neurons that released those glutamate molecules. That system requires energy, to drive the system and enable the pumping reactions to keep moving forward.

If the energy supply in a certain part of the brain or spinal cord is disrupted (such as, for example, by a stroke, cardiac arrest, or trauma that disrupts the blood or oxygen supply to brain or spinal tissue), the glutamate transport system will run out of energy, and the pumping reactions will stop. If that happens, excess glutamate molecules will begin to rapidly accumulate, in the watery fluid that fills the synaptic junctions between neurons. These free glutamate molecules will quickly begin to make the crisis even worse, because they will begin reacting again and again, in an uncontrolled and dangerously excessive manner, with the same glutamate receptors (including NMDA receptors, kainate receptors, and AMPA receptors) they had been binding to (as natural, useful, and essential neurotransmitters) before the crisis began. This will severely over-stimulate the receptor-bearing neurons, and it will drive them rapidly to a point of exhaustion, which can soon begin to cause nerve cells to begin dying, from over-exertion and exhaustion.

The ability of over-stimulation to begin killing nerve cells, in large numbers, arises from a crucial aspect of neuronal cell structure and behavior. The “resting” state for any neuron is actually a high-energy state, in which the neuron is perched on a high-energy plateau, where the neuron is (in effect) fully loaded, primed, and ready to fire off another nerve signal, as soon as it is activated. This is what allows an animal to respond rapidly to nerve impulses. A neuron reaches its “loaded and ready-to-fire” status by using energy to pump ions into and out of the neuron. This mainly involves pumping sodium ions, Na+, out of the neuron, to create a negative charge inside the cell; however, potassium, chloride, and calcium ions also play important roles in this process.

The ion pumping process continues until the neuron establishes a voltage gradient of roughly −90 millivolts (mV) across its outer membrane (different types of neurons establish voltages that range from about −70 to about −100 mV, when at rest). This voltage, across a neuron's outer membrane, drops substantially, during each “firing” or “depolarization” event. As soon as that happens, the neuron will begin using its energy supply to begin the ion pumping process again, as it tries to regain its high-voltage resting state, as quickly as possible, so it can be ready for the next arriving nerve impulse.

That constant process of ion pumping, by neurons, requires a great deal of energy; even though it weighs only a few pounds, the brain of an adult human uses roughly 20% of all the oxygen that is added to circulating blood, by the lungs. In addition, brain and spinal tissue do not (and cannot) keep any spare or reserve supplies of energy, or oxygen.

Therefore, if a crisis such as a stroke or cardiac arrest occurs, the glutamate transport system in the affected areas of the brain will slow down, because that transport system is not receiving enough energy supplies to keep driving it. When that happens, glutamate will quickly begin accumulating, in synaptic junctions in the brain. That glutamate accumulation will quickly make matters even worse, by triggering and activating the NMDA and other receptors on the neurons, even more frequently than before. This will rapidly cause the affected neurons to become severely stressed, and if the stress continues for more than a few minutes, it will begin to literally kill the exhausted and depleted neurons.

This type of glutamate-triggered neuronal death is called “excitotoxic” damage. The accumulation of excess glutamate, at NMDA receptors, is the major driving force behind this type of cell death.

Thus, it is known that glutamate, which is an essential neurotransmitter, takes on an entirely different role, during a crisis such as a stroke, cardiac arrest, severe epileptic seizure, or head trauma. In those types of crises, an essential neuroteansmitter becomes a toxin that plays a major role in worsening and aggravating the extent and severity of the permanent brain damage that is caused by the crisis.

It also should be noted, in passing, that acetylcholine neurotransmitters do not cause any similar damage, because the acetylcholine clearance mechanism is completely different from the glutamate transport system. When a molecule of acetylcholine is released by a neuron, it briefly binds to an acetylcholine receptor, and it is quickly released, so it will reenter the synaptic fluid. When that happens, the molecule is broken apart, by an enzyme called cholinesterase. This chemical reaction releases energy, rather than consuming or requiring energy to drive it. Therefore, since the cholinesterase reaction does not require any energy, to drive it, there is no danger of acetylcholine accumulation in the synaptic junctions in the brain or spinal cord, after a stroke or other crisis.

NMDA Receptor Blocker Drugs; Toxic Side Effects

After researchers learned that excessive activation of NMDA receptors plays a major role in aggravating the brain or spinal cord damage that is caused a number of important types of medical crises (and may also play an important role in aggravating some neurodegenerative diseases as well), researchers in numerous pharmaceutical companies and academic centers began developing drugs that can suppress activity at NMDA receptors. The hope was that if NMDA receptor blocker drugs could reduce excessive glutamate activity at NMDA receptors, they could reduce and minimize the severity and extent of brain or spinal damage, after a stroke or other trauma, or in various neurological disorders.

NMDA antagonist drugs were indeed shown to have impressive and highly promising neuroprotective properties, when tested in animal models that simulate stroke. However, when those drugs were subsequently tested in human clinical trials, they were shown to cause hallucinations and other effects that may mimic psychosis.

Subsequent tests in lab animals revealed that NMDA blockers, when administered at high dosages, have serious neurotoxic side effects. Even though they could reduce and minimize excitotoxic damage in a patient suffering a stroke or similar crisis, NMDA antagonist drugs were found to inflict a secondary type of injury to neurons in certain regions of the brain. These include a number of brain regions that can be regarded as “switchboard” or “traffic control” centers, which are highly important in the processing of sensory perceptions, thoughts, memories, etc.

At moderate doses of a highly potent NMDA antagonist (such as dizocilpine, also called MK-801, or phencyclidine, also called PCP), the neuronal stress typically is manifested initially in reactions such as vacuole formation (generally caused by vacuous swelling of mitochondria and endoplasmic reticulum) in cerebrocortical neurons, and in expression of so-called “heat shock” proteins (which are created as a cellular response to severe stress; these proteins were first detected when cells were immersed in very hot water, and then rescued). These types of stress reactions usually were reversible, if the exposure to MK-801 or PCP was only brief, but these stress responses progressed to irreversible damage and neuronal death, if exposure to an NMDA antagonist drug was prolonged.

Over the past decade, it has become evident that any NMDA antagonist drug which is potent enough to actually reduce brain damage, following a stroke or other crisis, will also cause unwanted and dangerous neurotoxic and psychotomimetic side effects, if administered at dosages high enough to reduce excitotoxic brain damage following a crisis. It has become clear that NMDA antagonist drugs pose enough of a risk of permanent brain damage that they must be carefully controlled.

Two facts can help illustrate these dangers. One fact is this: as of this writing, in February 2004, nearly 20 years after the first known and effective NMDA antagonist drugs were identified, numerous candidate NMDA blocker drugs were entered into human clinical trials, but each and every one of those drugs (with the sole exception of memantine) was subsequently abandoned. The FDA has approved, for public use and sale, only one drug that was known to have NMDA antagonist properties. That particular drug, memantine, is only a mild and relatively weak NMDA antagonist. It was not approved until the fall of 2003, and it was approved only for use in people who are already suffering from serious and irreversible brain damage, caused by Alzheimer's disease. The FDA has never knowingly approved any other NMDA antagonist, for any public use, even when limited to a “hospital use only” or “prescription only” basis under the control and supervision of a physician. The sole exceptions to date that have been approved by the FDA have been strictly limited to clinical trials only, for the express purposes of gathering more data as research continues.

The second fact which illustrates the dangers and risks of NMDA blocker drugs is this: the NMDA blocker drug called phencyclidine (or PCP) is sold illegally under the street name “angel dust”, and it is one of the most dangerous drugs ever created. PCP users experience vivid hallucinations and out-of-body sensations that last for hours, and they typically feel no pain of any sort, while they're high. That combination of hallucinatory and out-of-body experience, when added to the absence of any normal pain sensations, pose a dangerous and volatile combination, and PCP users often launch into violent and uncontrollable psychotic episodes, and may attack innocent people; PCP users have committed numerous gruesome murders, with no provocation of any sort. PCP once was used widely as an animal anesthetic, by veterinarians, but that use has dropped off sharply, partly because of the dangers PCP is now known to pose to the brains of the animals being treated, and partly because of the growing threat of drug abusers committing burglary, armed robbery, kidnaping, and other crimes in the offices of any veterinarians who continue to use phencyclidine.

Ketamine is one of the few NMDA antagonists that is currently being used in human medicine, as a surgical anesthetic. It was approved for this type of use roughly 40 years ago, long before it was known to have any NMDA antagonist properties (indeed, long before NMDA receptors were even discovered). It is difficult to know whether ketamine would be approved by the FDA or other regulatory agencies in other countries, if it were being submitted for approval today, and one can only speculate on that question.

However, despite the concerns over the neurotoxic side effects of NMDA antagonists, three factors apparently have convinced most surgeons and anesthesiologists that ketamine is sufficiently safe for normal use as a surgical anesthetic. First: as can be shown in cell culture tests, it is substantially less potent and aggressive, at binding to and blocking NMDA receptors, than MK-801 or phencyclidine. Second: it is used mainly for relatively brief periods, such as an hour or less, for surgeries such as setting a broken bone, or sewing up a wound (although in some cases it is still used for longer types of surgery). And third: anesthesiologists have realized that ketamine should be coadministered along with a benzodiazepine-type drug, such as diazepam (sold under the trademark VALIUM), which helps suppress unwanted excessive neuronal activity. This co-administration of a sedative-type drug (such as diazepam), along with ketamine, helps reduce the risk and severity of a transient form of disorientation and/or psychosis, commonly called a “ketamine emergence reaction”, which occurs among some surgery patients as they wake up after being treated with ketamine, if it was administered without a second drug such as diazepam.

However, to offset those reassuring factors, it also should be recognized that ketamine has become widely and illegally abused in recent years. It has become popular as a recreational drug of abuse, referred to on the streets and in dance clubs by names such as “Special K”. When used in that manner, it apparently causes euphoric and/or out-of-body sensations, mild to moderate hallucinations, etc.

Because ketamine is by far the most potent NMDA antagonist that is available for use by physicians and anesthesiologists today (dextromethorphan, the cough-suppressing agent, also has some minor level of NMDA blocking activity, but it is much less potent than ketamine), ketamine has been used on a number of occasions, in small-scale trials, for relief of severe intractable pain. However, to the best of the Applicants' belief, all such small-scale trials in the prior art have fallen into either of two tightly-limited categories.

One category involves use of long-term ketamine on patients in the advanced stages of a terminal disease, who will die fairly soon regardless of what is done to treat their pain. Examples of such treatments using ketamine are described in articles such as Klepstad et al 2001 and Kannan et al 2002. In that type of situation, the use of ketamine is humanitarian, and is designed solely to relieve suffering in a patient who will die soon, no matter what is done. Clearly, any concerns about possible neurotoxic side effects in that type of use are irrelevant, and are much less important than controlling pain, in a dying patient who otherwise would be in agony.

The second category of small-scale trials, which have tested ketamine for relief of chronic pain, used dosage regimens that were limited in various ways, such as by making the dosages intermittent rather than continuous. As examples, in the trials described in Fitzgibbon et al 2002, a single infusion of ketamine over 24 hours was followed by once-per-night pills; Rabben et al 2001 reported single intramuscular injections, followed by once-per-night pills; and, Mitchell 2001 reported a series of 21 infusions over a period of four months, which works out to an average of one relatively brief infusion every 6 days. Clearly, those regimens did not approach a dosage regimen as disclosed herein, which used continuous intravenous infusion for a span of multiple days.

It should be noted that certain legal developments are driving any type of sustained ketamine administration, by itself (in a form that can be called “mono-treatment”), even farther away from acceptable medical practice. The Applicants herein are aware of at least one potentially important lawsuit, alleging medical malpractice against a doctor who prescribed oral ketamine over a sustained period of time, in an effort to help a patient who was suffering from neuropathic pain. After that course of treatment had continued for some months, the patient became severely psychotic, and as of this writing is hospitalized in a mental institution. The family of that patient sued the physician who prescribed the ketamine, blaming the apparently permanent psychosis on the ketamine. As word of that lawsuit spreads through the medical community, it will serve as a potent warning to any physicians who might otherwise be tempted to prescribe ketamine, by itself, to patients suffering from various neurological disorders.

Finally, it should also be noted that during the 1980's, as soon as any new candidate NMDA antagonist became available to neurology researchers, those researchers (who were well aware, by then, of the crucially important role of NMDA receptors in CNS functioning) began testing these compounds, to evaluate their effects on nearly every conceivable type of neurological function that could be measured (including pain, sensory perceptions and processing, learning and memory, etc.). During the late 1980s and early 1990's, numerous published reports appeared, stating that NMDA receptor blockers could prevent or relieve neuropathic pain (e.g., Raigorodsky et al 1987; Aaronsen et al 1987; Woolf et al 1989; Davar et al 1991; Seltzer et al 1991; Yamamoto et al 1992; Mao et al 1992; Kristensen et al 1992; Backonja et al 1994).

Other reports from that same era also stated that NMDA blockers could interfere with learning, memory, and other forms of mental processing. As one example, Morris et al 1989 reported that when an NMDA antagonist was given to rats, it prevented them from being able to remember what they learned, later that same day, in a test using a tank of water with a submerged platform. However, treatment with the same NMDA blocker did not prevent rats who had already learned about the submerged platform, on previous days, from remembering where the platform was, and going to it quickly. Readers who are interested in the roles of NMDA receptors (which affect and even govern neuronal connections and networks) in learning and memory (and in unwanted ideations, delusions, compulsions, and other types of unhelpful or aberrant thoughts and memories) are referred to review articles such as Fuster 1997.

Interest in NMDA blockers as drugs that may be able to help treat and reduce neuropathic pain, or other neurological disorders, has never died out, and new articles on the subject appear sporadically. As one example, Mills et al 1998 described the testing of intravenous infusions of ketamine, for treating compulsive eating disorders (mainly in anorexic patients). The infusions were repeated from 2 to 9 times in various patients, at intervals ranging from 5 days to 3 weeks apart, using a 10-hour infusion period (20 mg per hour) for each session. Nine of the 15 patients tested reportedly had “prolonged remission” compulsive thoughts about food, diet, and weight.

Other examples which tested ketamine or various other NMDA blockers, on mental or psychological disorders, can also be found in the literature. However, none of them used or even approached the combined-drug treatment regimens disclosed herein.

“Safener” Drugs

When evaluating the prior art concerning NMDA antagonist drugs, it should be noted that a number of drugs which have been called “safener” drugs can be administered, along with NMDA antagonist drugs, to reduce or eliminate the neurotoxic side effects that are caused by potent NMDA antagonists (such as MK-801 or phencyclidine), or by large doses of ketamine.

A clear, unambiguous, and readily-evaluated standard to determine whether some drug is or is not a “safener” drug (as that term is used herein) can be generated fairly easily, using rats, in tests that use an NMDA antagonist drug called MK-801. This compound is the maleate salt of dizocilpine, and it is highly potent in blocking activity at NMDA receptors. It also is highly selective for NMDA receptors, and has no known interactions with any other types of neuronal receptors. Therefore, MK-801 is widely used and well-known in research on NMDA receptors. Phencyclidine (abbreviated as PCP) is also used in such research, since it is also very potent at blocking NMDA receptors, but it may be somewhat less selective, and it also poses serious risks of theft and abuse, so MK-801 is the preferred drug for such testing.

When injected intraperitoneally into rats, at one-time dosages of about 0.3 mg/kg or greater, MK-801 will cause a vacuole response in various regions of the rat brain, including the posterior cingulate/retrosplenial cortex. If the duration of NMDA receptor blockade is prolonged beyond about 12 hours, either due to a large single dosage or repeated smaller dosages, the brain damage becomes permanent, and additional brain regions become involved. This is shown by the necrosis and death of neurons, which can be counted by using staining techniques. These types of assays, and the types of neuronal damage that can be found after treatment by a potent NMDA blocker such as MK-801 or phencyclidine, are described in more detail in articles such as Olney et al 1991, Fix et al 1993 and 1994, and Corso et al 1997.

Accordingly, if a candidate drug can substantially reduce or entirely block that type of vacuole response, in rat brains, caused by drugs such as MK-801 or phencyclidine, then that candidate drug falls within the definition of “safener” drugs as used herein.

It should be noted that vacuole formation can be used as a convenient indicator of neurotoxic side effects of NMDA antagonists, since vacuoles can be observed and counted, fairly easily and inexpensively, using a light microscope after relatively simple tissue fixation and staining steps have been carried out. By contrast, evaluating and quantifying other types of neurotoxic side effects that are known to be caused by NMDA antagonist drugs requires more time-consuming and expensive procedures and reagents. As one example, analysis of whether “heat shock” proteins or mRNA were expressed in the vulnerable regions of the brain of a treated animal can be carried out, and indeed has been done, to provide confirmation that neuronal vacuoles in certain regions are a valid indicator of severe stress in the brain. However, those types of assays require specialized and expensive reagents, and they are more difficult, time-consuming, and expensive than simply using a light microscope to count the numbers of vacuoles in stained tissue sections from selected brain regions.

It also should be noted that the term “safener” was adopted from a different but analogous practice, used by herbicide manufacturers and farmers. If that practice in the realm of herbicides is understood, it may shed more light on this effort to import the concept of safener technology from herbicide chemistry, into pharmaceutical chemistry.

Briefly, a number of herbicide safeners are known that can be applied to specific types of crops. This type of safener application is usually done by coating the safener compound onto seeds, or by spraying a field of crops with a safener, usually about a week before a potent herbicide is sprayed on the same field of crops. The safener compound will trigger a response in the crops, usually involving overproduction of a certain enzyme that will detoxify a certain herbicide. This leads to the safener-treated crops developing a higher level of tolerance for (and resistance to) a certain herbicide. After this type of treatment has been applied, the weed-killing herbicide can be applied to the “safened” crops at a heavier dosage, which can achieve greater and more effective killing of any weeds, without damaging the crop plants that were given greater resistance, by the safener.

Nearly 20 safener-herbicide combinations are used commercially, and can be located fairly easily in an Internet search. As examples, safeners such as dichlormid or benoxacor can be used, in corn and sorghum, to increase the resistance of those particular crops to acetanilide or thiocarbamate herbicides.

That digression into herbicide terminology was included in this description, because it shows that the safener approach has indeed been developed and used effectively, commercially, and successfully, in a way that allows one type of chemical to reduce the toxicity of another type of chemical.

In a different but analogous manner, safener drugs, as disclosed and used herein, can protect a mammalian CNS (or at least certain vulnerable portions thereof) against the neurotoxic side effects of NMDA antagonist drugs. This can allow the safe and effective use of NMDA antagonist drugs, at dosages that are higher and more prolonged than could be tolerated safely by patients, or approved by government agencies.

Drugs that have been shown to be safener drugs (using the benchmark test specified above, involving reduction or prevention of the neurotoxic side effects of MK-801, in tests on rats) can be divided into several categories, depending on which neurotransmitter system they affect. Briefly, they include:
(1) drugs that suppress activity at the muscarinic m3 class of acetylcholine receptors, as described in U.S. Pat. No. 5,034,400 (olney 1991);
(2) “direct-acting” GABA agonist drugs, as described in U.S. Pat. No. 5,474,990 (Olney 1995);
(3) alpha-2 adrenergic agonists, as described in U.S. Pat. No. 5,605,911 (Olney et al 1997);
(4) drugs that suppress activity at non-NMDA (kainate and/or AMPA) receptors, as described in U.S. Pat. No. 5,767,130 (Olney 1998); and,
(5) drugs that activate the 5HT-2A (but not the 5HT-2C) subclass of serotonin receptors, as described in U.S. Pat. No. 5,902,815 (Olney et al 1999).

However, in evaluating this current invention, it should be noted and recognized that even though these “safener” drugs began to be disclosed more than 10 years ago, not a single pharmaceutical company anywhere in the world has chosen to develop, obtain approval for, and market, a combination of an NMDA antagonist drug together with a safener agent. Prior to this invention, neurology researchers have known for at least a decade that NMDA receptor blocker drugs have good potential for reducing the severity of brain damage caused by a stroke, cardiac arrest, or other crisis. Researchers have also known, for more than a decade, that safener drugs are also available, which can reduce or completely prevent the neurotoxic side effects of those NMDA receptor blocker drugs. However, no pharmaceutical company has ever chosen to pursue that line of research and development, involving a combination of an NMDA blocker drug along with a safener drug.

NMDA Blockers With Inherent Safening Activity

On the subject of safener drugs, it should be noted that a few compounds have been identified that can suppress activity at NMDA receptors, and that also appear to have some degree of inherent safening activity, at least when tested in animal models.

The two main known compounds that fall into this category are closely related, since they both come from an African plant with the species name Tabernanthe iboga. The compounds are called ibogaine (which apparently is more active, and has received more attention) and ibogamine. Extracts from the plant have long been known by native users to cause various neurological effects; curiously, even though they can cause hallucinations, they can also enable a hunter to remain remarkably still, for hours at a time. These different and apparently inconsistent traits (as well as the inherent safener activity of ibogaine) are presumed to be due to the fact that ibogaine is active not just at NMDA receptors, but also at sigma receptors as well, and is also suspected of being active at serotonin receptors also.

Ibogaine has been studied in various ways, after several reports and patents appeared in the 1980's, asserting that it could help drug addicts break free of their addictions. Relevant patents, all issued to Howard Lotsof, include U.S. Pat. No. 4,499,096 (issued in 1985, on heroin addiction), U.S. Pat. No. 4,587,243 (1986, on cocaine and amphetamines), U.S. Pat. No. 4,857,523 (1989, on alcohol), U.S. Pat. No. 5,026,697 (1991, on tobacco and nicotine), and U.S. Pat. No. 5,152,994 (1992, on multiple drug dependencies).

A formal evaluation in a clinical trial was commenced in the 1990's, under the supervision of a University of Florida neuroscientist named Deborah Mash, but that trial was terminated when one of the patients died. Reportedly, at least one under-the-radar clinic still operates somewhere in the Caribbean, where professionals with money who are addicted to drugs fly down and get a one-day treatment. Along those lines, it should be noted that one of the main appeals of ibogaine, as a potential agent for treating drug addiction, is the claim that a single treatment with ibogaine can create a lasting alteration in a addict's cravings for addictive drugs.

For a truly interesting short account of ibogaine, how it came to be stuck in legal and scientific limbo, and the conflicts and lawsuit that arose between Lotsof and Mash, readers are referred to an article (“Molecule of the Month”, September 2002) that can be downloaded from www.chm.bris.ac.uk/motm/ibogaine/ibogainej.htm. Those who want a purely scientific review are referred to Popik et al 1998.

Now that a protocol has been established which has shown that an NMDA receptor blocker drug, when administered in a certain way (using dosages that cause speech slurring but not unconsciousness, over a span of three to five days continuously), can reliably generate lasting improvements in patients suffering from severe neurologic disorders, ibogaine and any of its analogs may merit reevaluation, at speech-slurring but non-hallucinogenic dosages, in clinical trials to evaluate their efficacy for use as described herein.

Accordingly, certain claims set forth below refer to “an NMDA antagonist having inherent safener activity”. Those claims are intended to include ibogaine and ibogamine.

Two other known NMDA blockers that are believed to have inherent safener activity include ifenprodil and eliprodil, described in Contreras et al 1990 and Carter et al 1988. As with ibogaine, their inherent safening activity appears to be due to their activity at sigma receptors.

It should also be recognized that certain types of drugs that are known as anti-cholinergic drugs (such as procyclidine and ethopropazine) also have some level of NMDA receptor blocking activity. While such drugs, in their current known form, do not have sufficiently strong NMDA antagonist activity to render them preferred candidates for early evaluation for use as disclosed herein, two points should be noted. First: anti-cholinergic agents (especially those that block activity at the m3 subclass of muscarinic acetyl-choline receptors) can offer safening activity, when coadministered with potent NMDA blockers. Second: mild suppression of acetyl-choline receptors, in a patient's central nervous system, may be useful in helping that patient rapidly reach a condition referred to below as an “improved stable neural homeostasis”. This possibility merits evaluation, fairly early during the course of any evaluative tests that are carried out as described herein. If it is shown to provide improved results in treating one or more particular types of neurological disorders, it can thereafter be incorporated into any such treatments, as an enhancement, at the discretion of the treating physician.

Accordingly, a number of “inherently safened” NMDA blockers having varying levels of potency are already known, and others can be discovered or created, by creating and screening analogs and derivatives of those known drugs, using well-known cell culture tests, followed by animal tests of compounds that perform well in the cell culture tests. Accordingly, any such already-known or hereafter discovered NMDA antagonist having inherent safener activity can be tested, to evaluate its suitability for use as described herein.

Use of NMDA Blockers and Safeners to Treat Neuropathic Pain

As described in a number of articles cited above from the late 1980's and early 1990's, neurology researchers have known for well over a decade that NMDA receptor blockers have good potential for being able to prevent or treat neuropathic pain. And, as noted above, they have also known for more than a decade that safener drugs are available which can reduce or prevent the neurotoxic side effects of those NMDA receptor blocker drugs.

However, despite the fact that both of those components have been known for well over a decade, no pharmaceutical company has ever chosen to actively pursue clinical trials and potential commercialization of a combination of an NMDA blocker drug along with a safener drug.

That situation is likely to change before long, since the Inventors herein have recently discovered and shown that a combination of sustained-dosage ketamine, when coadministered with clonidine (a safener drug which is an alpha-2 adrenergic agonist) can provide remarkably effective treatments for certain types of chronic neuropathic pain that, until now, have been effectively untreatable, and incurable. As described in utility patent application Ser. No. 10/373,433, entitled “Prolonged Administration of NMDA Antagonist and Safener Drug to Alter Neuropathic Pain Condition,” the ketamine-clonidine combination, when administered continuously for a span of up to five days in a row, at a dosage that is titered to cause slurred speech but no loss of consciousness in a patient being treated, has been able to provide, for most patients treated to date, what appears to be a complete and total cure (or at least a major reduction) for their intractable chronic pain. Since it has not yet been published, a printed copy of the text of that application is enclosed herewith. Its contents are incorporated herein by reference, as though fully set forth herein.

That application and its claims are focused upon and limited to the treatment of chronic and/or neuropathic pain. However, after seeing and analyzing the results of those treatments, the Inventors herein have realized that the drugs and treatment regimens that were initially developed and tested for treating neuropathic pain, can also treat other neurologic disorders as well, beyond the realm of chronic pain.

Indeed, the process of testing, tweaking, and optimizing these drug combinations, in tests on patients who suffer from severe neuropathic pain, ended up providing the Inventors with an excellent and powerful test system, which allowed them to gather highly useful data in a rapid and effective manner. Patients who are suffering from severe and chronic pain are not reticent about discussing it honestly with a treating physician. They can and will give an immediate, accurate, and honest appraisal of their pain condition and status, whenever asked (and frequently when not asked, if they think any changes may be occurring). By contrast, when people who are suffering from neurologic disorders that have mental, behavioral, or emotional components are asked to disclose the truth about what is going on inside them, they often have private and personal desires and reasons for concealing the truth. Except for people who are truly psychotic or delusional, most people who have struggled to cope with a severe neurologic disorder have learned, through years of intense unhappiness and hardship, to keep their private thoughts and beliefs quiet, secret, and hidden, whenever possible, and they often become quite skilled at doing so.

Therefore, as events unfolded over time, the evaluation, tweaking, and development of treatments that are potent and effective in creating “improved stable neural homeostasis” alterations, in patients suffering from chronic pain, provided an ideal system for developing and testing drug combinations and regiments that can, indeed, alter a person's neural homeostasis on a lasting and stable basis. Therefore, the results obtained by testing patients who suffer from neuropathic pain have established a confidence level that would have required multiple years, and hundreds or even thousands of patients, if the only patients who could be tested were suffering from behavioral or emotional disorders that, in many cases, might cause treated patients to be less than candid, honest, or fully competent in their reports of progress and changes (or the absence thereof).

Because of factors discussed below, the treatments disclosed herein (using sustained coadministration an NMDA blocker drug at a “maximum tolerated dosage” that causes speech slurring but no loss of consciousness, and a safener drug that can prevent or minimize the neurotoxic side effects of the NMDA blocker drug) are capable of providing what is, in effect, a “reset switch” for the central nervous system (CNS) of a human or other mammal.

In many cases, an impaired CNS that is suffering from a severe neurologic disorder can be thought of as being trapped and held in an unwanted position that mathematicians would call a “local minimum”. When a curve is plotted on graph paper, a “local minimum” is a spot in the curve that is lower than its immediate surroundings, but higher than other locations on the curve. As one example, the shallow rounded depression on top of a golf tee is a “local minimum”. It can hold a golf ball, in a quasi-stable condition, for weeks or months, if nothing moves the golf ball. However, the ball would be in a lower and more stable position, if it could be nudged off of the golf tee, allowing it to drop onto the surrounding grass.

As another example, using larger terms, if a hiker has descended into the crater of a volcano, that crater is a “local minimum” location. The hiker will have to climb up the walls of the crater, before he can travel down the side of the mountain, to a much more stable condition down below.

In an analogous manner, many people who are suffering from neurologic disorders that cannot be adequately cured and solved under the prior art (such as drug, alcohol, or tobacco addiction, severe depression unrelated to physical pain, an obsessive-compulsive disorder, etc.) are trapped in “local minimum” spots. However, instead of being in a gentle sloping depression that can be escaped easily, these cage-like depressions are analogous to the “pot-bunkers” that are often found on championship-caliber golf courses. Pot-bunkers are deliberately designed as small and deep sand traps, with high and steep walls that usually are “crowned” with a heavy layer of unmowed, overhanging grass. They are extremely difficult to escape, and that makes them interesting challenges, among skilled golfers who like to play on difficult courses.

The analogy must end there, because there is nothing that even remotely resembles an “interesting challenge”, for someone who is struggling and suffering terribly, while trying to break free of neuropathic pain, drug addiction, or any other severe neurologic disorder. Most people simply cannot escape from that version of hell, without help.

Prior to this invention, no one has successfully managed to find or create a pharmaceutical intervention that can calm and quiet the central nervous system, in a way that can enable the nervous system to repair and heal itself in a way that will provide a lasting, stable, and possibly even permanent improvement (which is completely different from palliative relief that will only make the problem worse over the long run, if continued, using short-term agents such as pain-killers or tranquilizers).

To understand how this pharmaceutical intervention works, and how it can enable a badly impaired nervous system to move to a better condition without unacceptably high risks of causing the nervous system to go wandering off into areas that might be even worse and more dangerous, it is necessary to consider a biological condition called homeostasis.

Homeostasis: Changing and Responsive Equilibrium

The concept of “homeostasis” needs to be understood, to grasp this invention and to begin to understand what the drug treatments disclosed herein can achieve, when used to treat patients suffering from severs neurologic disorders that do not relate to pain (such as, for example, drug or alcohol addiction, obsessive-compulsive disorders, long-term clinical depression, etc.).

“Homeostasis” is a crucial and fundamental biological process and principle, discussed in any textbook on physiology (e.g., page 4 of the Textbook of Medical Physiology by Guyton and Hall, 9th edition, 1996). It refers to the maintenance of consistent and properly functional conditions, within a “steady-state” set of boundaries and constraints, in an organism.

This does not mean that the processes inside an organism remain static and unchanging, because nothing in life is static and unchanging. Instead, it implies and requires that: (i) all essential conditions (such as body temperature, blood flow rates, etc.) must remain within a set of reasonable and proper boundaries and constraints; and, (ii) when a disruption occurs, the organism and each of its components (including its internal organs, nervous system, etc.) will respond and adapt to the disruption, in a manner that brings the organism back toward a stable ongoing equilibrium.

As one example, if an animal eats a large meal after a long period of hunger, the sudden entry of a large quantity of food, into an empty digestive system, will provoke a complex set of responses and changes, mainly but not exclusively involving the stomach and intestines. The effects of those adaptive responses to the sudden and rapid ingestion of a large bolus of food will be to ensure that the animal's body temperature, blood flow rates, and other biological parameters all remain relatively constant, stable, and reliable, within proper operating limits.

In a similar manner, if an animal becomes cold, it likely will begin shivering, which is an adaptive response. Shivering is a muscle activity that generates warmth, by using muscles to burn stored energy at accelerated rates.

Those and thousands of other types of responsive changes, which allow an animal to adapt and modify itself in ways that respond to changes and challenges in its environment, are all designed to help sustain “homeostasis” within an animal's body.

Another way to grasp the concept of homeostasis is to think in terms of a “set-point”, which generally refers to a targeted and desirable set of operating conditions for a machine or system, either mechanical or biochemical. As an illustration, an automobile engine will run best (most efficiently, generating good torque and horsepower, etc.) if it can run within some range of revolutions per minute (RPM). If an automobile engine is forced to run at a speed that is either too fast or too slow, it will not run properly.

The concept of “set-point” can be understood and applied to humans and other animals, by considering what happens when a bone heals, after it has been broken. A broken bone does not have a brain, and it does not have its own awareness or consciousness (apart from the sensations of pain that are generated by the animal's overall nervous system). Nevertheless, the cells and tissues in and around a broken bone somehow “know” what the proper set-point for that bone was, and is, and ought to be. When that set-point is disrupted by a break in the bone, the cells that secrete hydroxyapatitie (the calcium-phosphate mineral that forms hard bone) are somehow “awakened”, and are instructed to become active once again, in ways that will cause the cells to begin depositing new layers of mineral across the newly-exposed surfaces where the bone was broken. Those cells somehow “know” how to respond and adapt to the break, in ways that will move the affected tissues back toward the “set-point” location, where everything was connected properly and working smoothly.

The pharmaceutic interventions that have been developed by the Inventors herein for treating neurologic disorders (either neuropathic pain, as described in patent application Ser. No. 10/373,433, or non-pain disorders such as drug addictions or obsessive-compulsive disorders, as disclosed in this current application) are comparable to placing a splint over a limb that has suffered a broken bone. The splint does not cure or heal the bone; nevertheless, it can give a broken bone a greatly improved chance to heal properly, without leading to even more serious disorders that can arise if a broken bone knits back together in an awkward and incorrect manner. By removing the stresses, jostling, disruptions, and “re-breakages” that would otherwise harm a damaged bone that is trying to heal and repair itself, a splint can make all the difference between the bone being able to heal properly, or not being able to heal properly. A splint does not provide the minerals that will knit a bone back together; nevertheless, it plays a crucial role, by enabling the normal and natural homeostatic processes that are already present, in that limb, to move the affected limb with its broken bone back to a normal, natural, healthy set-point.

This invention takes that same general concept and approach, and adapts it to enable a form of neurologic healing that uses homeostasis (i.e., an adaptive process that seeks ongoing equilibrium and stability) and set-points (stable and secure conditions or locations, which a biological system will normally and naturally incline toward, and move toward, if given a chance to do so).

This invention compiles and assembles, for the first time, several distinct lines of medical and pharmaceutical research into a coordinated package. This coordinated package appears to be capable of providing, quite literally, a “reset switch” or “circuit breaker” for the central nervous system, in humans who are suffering from severe neurologic disorders other than chronic or neuropathic pain.

This combined drug treatment, when administered continuously and without any cessation or letup over a span of about 5 days, has created remarkable and even extraordinary results, by providing what appear to be lasting and even permanent cures for some of the worst and most intractable types of chronic pain that are known to medicine. Based on those apparently stable improvements and permanent results, the Inventors herein have realized and concluded that these treatments can enable a human central nervous system to adjust and improve its own condition, in a manner that can return it to (or at least move it closer to) the same stable, healthy, pain-free set-point that had been functioning properly for years, before the onset of the chronic pain disorder.

Because of the way NMDA blocker drugs work (i.e., by reducing excitatory activity, at the most important type of excitatory receptor in a mammalian nervous system), these drugs offer what is, in effect a period of “quiet rest” for a damaged nervous system. Most patients tested to date were suffering from an extremely difficult and incurable pain condition known as “reflex sympathetic dystrophy” (abbreviated as RSD). The root word “troph” refers to nourishment, and “dystrophy” indicates a lack of nourishment. This neuropathic condition was given that name, because it is so severe that it gradually causes the destruction, atrophy, and loss of muscle tissue.

Recently, the syndrome known as “reflex sympathetic dsytrophy” was renamed as “complex regional pain syndrome, type 1” (CRPS-1). That renaming did not reflect any lessening of the severity or incurability of RSD; instead, it reflected the realization that RSD patients were not being helped when doctors told them, “It's incurable, the word sympathetic is a cruel joke, and you're going to slowly waste away, in severe pain.” Instead of giving that bleak message to those patients, the phrase “complex regional pain syndrome” implied a different theme, along the lines of, “It's complex, and we don't have a cure, but we're working on it, and at least it hasn't spread to your whole body, so don't give up hope.”

The “Type 1” designation in CRPS-1 refers to cases of CRPS that do not have any known causative factor, such as an injury or infection (the medical term for problems with no known causes is “idiopathic”). Therefore, CRPS-1 cases are medical mysteries. That renders them even more difficult to diagnose and treat than Type 2 cases, which were triggered by an injury, infection, or other problem that was known and could be treated.

Since RSD (CRPS-1) was effectively incurable under the prior art, and had no known treatments other than heavy dosages of morphine, Oxycontin(™), or other pain-killing drugs and sedatives, the Food and Drug Administration was willing to approve experimental treatments that offered at least some hope of at least some relief to sufferers, despite the knowledge that such treatments could pose serious and even severe risks to the patients who were being treated and tested.

Various physicians around the world (including in Germany, Russia, and Australia) had been treating RSD (CRPS-1) patients with intravenous ketamine, for several days in succession, with mixed results. Some of these treatments involved ketamine dosages that effectively rendered a patient unresponsive or even comatose, for a period of roughly a week at a time; other treatments involved lower dosages, which caused observable manifestations (such as slurred speech) that stopped short of rendering the patient unconscious. Although some improvements were reported by some patients who were treated in that manner, those treatments fell short of being ideal or even satisfactory.

One of the coinventors herein (Correll, a physician) was involved in those tests, in Australia, using ketamine “mono-therapy” (i.e., without also administering a safener drug, such as clonidine). One of the other coinventors (Harbut, an anesthesiologist) went to Australia for a month, witnessed and participated in some of Correll's treatments, and realized that the results were positive to a point of being tantalyzing and suggestive, without being entirely adequate or satisfactory. That coinventor subsequently returned to the United States and obtained funding for further study of ketamine mono-therapy, in the hope that it could be improved and optimized to make it more effective and reliable, by means such as developing improved dosage regimens that could be calculated based on medical factors involving each patient.

During the course of one of those tests, a patient's blood pressure began to increase to undesired levels. In response, the anesthesiologist began to administer clonidine, a standard and conventional treatment for controlling high blood pressure. The patient then reported a substantial improvement in his pain reduction, which was surprising. The anesthesiologist then began researching the scientific, medical, and patent literature on the subject, and soon discovered prior work done by the third inventor (Olney, a neurology researcher who specializes in animal models) on the subject of safener drugs that can reduce or prevent the toxic side effects of NMDA blocker drugs such as ketamine.

After bringing those various skills (and various pieces of the puzzle) together, and after seeing the results gathered by Harbut from additional tests using controlled combinations of ketamine and clonidine on RSD (CRPS-1) patients, the coinventors realized they had discovered and created a safer, more effective, and longer-lasting treatment for a severe and previously incurable form of neuropathic pain, than had ever previously been known to anyone. As mentioned above, that discovery is described and claimed in U.S. patent application Ser. No. 10/373,433.

However, the Inventors did not stop there. After pondering how that type of treatment, administering a combination of certain drugs having known activities and effects for a continuous period lasting several days in a row, could enable treated patients to make huge strides toward regaining a healthy and pain-free “set-point” they had enjoyed for years before the onset of their debilitating disorders, the Inventors realized that, because of the way the selected drugs function and interact, inside the brain and spinal cord, similar treatments can also enable major neurologic improvements, in at least some patients who are suffering from neurologic disorders that do not involve physical pain.

One of the insights that led to expansion of the pain treatments into other types of neurologic disorders was the realization that, for someone who is suffering from an addiction or mental disorder, “quiet rest” simply cannot be provided or achieved, without a direct and powerful pharmaceutical intervention. As one example, if a person's nervous system has become addicted to cocaine or heroine, “quiet rest” isn't quiet at all; instead, it is tormented by cravings, longings, frustrations, and disappointments. Similarly, if a person is suffering from severe depression, “quiet rest” isn't restful; instead, it merely adds more days to an endless series of wasted, useless, and miserable weeks and months. Therefore, people in that type of condition need help, and intervention, from an outside source.

Finally, one additional factor helped trigger the realization and understanding of what is really going on, inside a patient's brain and nervous system, when that patient receives a sustained NMDA receptor blocker treatment. That factor involves a comparison of this type of therapy, to electroconvulsive therapy, as discussed below.

Comparison to Electroconvulsive Therapy

“Electroconvulsive therapy” (abbreviated as ECT) is the current name given to what formerly was called “shock therapy”. In this type of treatment, two moderately large paddle-type electrodes are typically placed on opposite sides of the patient's brain, flanking the forehead region, and a large surge of voltage and current are passed through the patient's head. The patient has a convulsion, is rendered generally unconscious or at least stuporous, and then recovers for a period of several days, while his brain works it ways back into working order.

This type of therapy, which was first developed in the 1800's, when no other treatments were even remotely available for treating severe mental illnesses, is comparable to using a sledgehammer, to break apart concrete. It is a crude, heavy, blunt instrument, with no subtlety, and no ability to be targeted at, or limited to, any particular region of the brain, or any particular class of neuronal receptors.

Nevertheless, it is sometimes remarkably effective, in treating certain types of extremely severe neurological disorders that will not respond to drug therapy, psychoanalysis, or other treatments. Most such cases in current practice involve intense and severe depression, where thoughts of suicide have risen to a point of deeply concerning friends and family. Prior to about 2002, ECT was also used fairly commonly (and fairly successfully) to treat extreme cases of obsessive-compulsive disorder (OCD); however, recent advances in drug therapy for OCD patients have reduced the number of OCD cases that cannot be treated by drug therapy and that lead to ECT.

ECT is still used today, in modern psychiatric practice, to an extent that probably would surprise most laypersons. A medical journal, the Journal of Electroconvulsive Therapy, continues to be published, with articles containing quotes such as, “Many patients with major psychiatric disorders who are severely ill, medication-resistant, or medication-intolerant respond more reliably and quickly to a course of electroconvulsive therapy” (from Rabheru et al 1997).

In essence, ECT offers a way to “reshuffle the deck” without killing the patient. The shock-wave surge of electro-convulsive current that passes through a person's brain will randomly disrupt and/or disconnect uncountable numbers of synaptic connections, between neurons in the brain.

The hopes and goals of this type of random disruption of neuronal connections and networks, inside the brain, are two-fold. First, it is hoped that large numbers of unhelpful and unwanted neuronal connections, which had been contributing to making a patient's psyche, attitudes, and outlook on life a miserable ordeal, will be among the connections that are disrupted, and disconnected, by the electroconvulsive shock-wave as it travels through the brain. Second, it is hoped that when the neurons in the brain set about the task of creating new connections and networks, to replace the ones that were disrupted by the shock-wave, the new connections can be formed in ways that will not lead to the same severe problems that existed before the treatment was performed.

In other words, passing an electroconvulsive shock-wave through the brain of a patient is a way of using a “blunt instrument” to rapidly force a patient's brain into an altered neural homeostasis. The hope is that, since the patient was utterly miserable (to a point of seriously contemplating suicide) before the treatment, any alteration is likely to be an improvement, compared to the pre-treatment condition that the patient was suffering.

It must be noted that the non-selective, intensely disruptive nature of an electroconvulsive shock-wave, passing through the brain of a patient, inevitably causes unwanted side effects. Disorientation and memory loss are suffered by all ECT patients, as an inevitable byproduct of the process, and many ECT patients suffer from additional problems as well.

The crucial point is this: despite its crude, heavy-handed, and non-selective nature, electroconvulsive therapy is a powerful and effective way of driving the patient's brain into an altered (and hopefully improved) form of neural homeostasis. And, it actually works, in providing effective and useful treatments for certain classes of patients who are suffering from severe neurological disorders, such as intense depression to a point of seriously contemplating suicide, and obsessive-compulsive disorders that have become so severe that they are ruining a person's life and could not be treated by drugs (until recently, as noted above).

As mentioned above, electroconvulsive therapy was tested and used in the 1800's, long before the advent of modern medicine, and long before glutamate or NMDA receptors were known to exist inside the brain. Nevertheless, even though its practitioners did not understand neurology, and did know how or why it was working on a cellular level, they understood full well that its effect and goal was to create what would be referred to today as an “altered neural homeostasis”. The “reshuffle the deck” concept was recognized and understood, decades ago; physicians knew they were introducing uncontrollable disruptions and changes into the brains of people suffering from serious problems, in the hope that the brain could heal itself, and reach a better place than before, after the deck had been reshuffled.

Since that type of treatment, although drastic and non-selective, can indeed help at least some classes of patients who are suffering from severe neurological problems, it validates and confirms the underlying concept of this invention. This invention involves a more selective, gentle, and benign yet highly potent and powerful way to create a stable and lasting alteration, in a patient's neural homeostasis.

Indeed, it is hoped and anticipated that this new form of benign and selective yet powerful and effective therapy will completely displace electroconvulsive therapy, and render it totally obsolete.

Finally, on the subject of chronic depression that is not related to chronic or neuropathic pain, it should also be noted that various research teams have tested various ketamine dosage regimens, to determine whether such treatments can help control and reduce depression. The regimens that have been tested in such reports do not resemble or even approach the regimens disclosed herein; as examples, Berman et al 2000, Kudoh et al 2002, and Yilmaz et al 2002 all report transient relief of depression, following a single one-time dosage of ketamine. Despite those and other potentially promising reports, ketamine treatment is not used as a recognized or accepted therapy for depression, and the Inventors are not aware of any tests using ketamine administration that lasted for several days in succession. A recent review that summarizes tests of ketamine (and various other agents on depression) is provided in Sanacora et al 2003.

Accordingly, one object of this invention is to disclose a strong and powerful pharmaceutical intervention, for treating people who are suffering from serious neurological disorders that cannot heal themselves, in a manner that can enable a patient's central nervous system to use its own homeostatic processes to move toward a stable, long-lasting, and in many cases even permanent improvement in the neurological disorder.

Another object of this invention is to disclose strong and powerful pharmaceutical interventions that can provide lasting and stable improvements in various types of severe neurologic disorders. Candidate disorders that merit evaluation, to assess the efficacy of such treatments for such disorders, include drug addictions, alcoholism, high-risk or pathological behavior such as compulsive gambling, severe chronic depression or bipolar disorder, obsessive-compulsive disorders, and various types of behavioral, emotional, and/or sexual disorders.

Another object of this invention is to disclose that, if a drug that suppresses activity at the NMDA subclass of glutamate receotrs is administered at a “maximum tolerated” dosage (which must be titered individually, for each patient) over a sustained period of time (such as five days, continuously), to a patient who is suffering from a severe neurologic disorder, then the intervention by the NMDA blocker drug can help enable the patient's central nervous system move closer to an “improved stable neural homeostasis”, which will place the patient in a better, happier, and healthier condition.

Another object of this invention is to disclose that NMDA antagonist drugs, if administered at or near a maximum tolerated dosage over a sustained period of time, can help a patient who suffers from a serious neurologic disorder achieve a stable and lasting improved neural homeostasis.

Another object of this invention is to disclose that NMDA antagonist drugs, if administered at or near a maximum tolerated dosage over a sustained period of time, can help treat and reduce severe neurologic disorders that have behavioral and/or emotional manifestations, such as alcohol, tobacco, or drug addiction, compulsive gambling, shoplifting, or other risk-taking, eating disorders, etc.

Another object of this invention is to disclose that these types of medical interventions, using sustained maximum tolerated dosages of an NMDA antagonist drug to create a lasting and stable alteration in a patient's neural homeostasis, can be rendered safe and effective, if a second drug which has “safening” activity (such as an alpha-2 adenergic agonist drug, such as clonidine) is also administered along with the NMDA antagonist drug.

Yet another object of this invention is to disclose that these types of medical interventions, using sustained maximum tolerated dosages of ketamine to create a lasting and stable improvement in a patient's neural homeostasis, can be rendered more effective, if magnesium and/or an agent that inhibits the metabolic degradation of ketamine is also administered, along with ketamine and a safener drug.

These and other objects of the invention will become more apparent through the following summary, drawing, and description.

### DETAILED DESCRIPTION

As summarized above and described in the claims, a method is disclosed for treating a neurologic disorder, other than chronic or neuropathic pain, comprising coadministering to a patient: (i) at least one NMDA receptor antagonist drug, at a dosage-time combination that has been shown in human trials to be effective in creating stable and long-term neurological alterations in treated patients; and, (ii) at least one safener drug that has been shown, using in vivo animal tests, to reduce neurotoxic side effects caused by MK-801 when administered without an accompanying safener drug.

An exemplary NMDA receptor antagonist (or blocker) drug is ketamine, and an exemplary safener drug is clonidine. A suitable dosage-time regimen can be created most conveniently and reliably by: (i) titering the dosage of the NMDA receptor blocker until the patient's speech becomes somewhat slurred, while staying below a dosage level that would cause a patient to become stuporous or lose consciousness; and, (ii) sustaining that dosage for period of several days in succession (including overnight administration, while the patient is asleep), such as preferably at least about 3 days, up to about 5 days. To satisfy the claim language, dosage-time combinations do not need to be proven efficacious in all patients tested; however, a dosage-time combination that is being considered for use in treating any type of neurologic disorder that will not allow prompt and reliable reporting or other evaluation generally should use a dosage-time regimen that has been shown effective in a large majority of patients tested, unless a treating physician concludes that a somewhat lower dosage-time combination is preferable for some particular patient, based on a careful evaluation of his or her condition or history.

If blood concentrations are being monitored, preferred treatments establish and sustain a blood concentration of at least about 200 nanograms of ketamine, per milliliter of blood plasma, for a period of at least 24 hours. Blood concentrations of at least about 250 nanograms of ketamine, per milliliter of blood plasma, for a period of at least 40 hours are likely to be even more preferable, for most treatments.

The drugs can be administered orally if desired; however, intravenous administration is generally preferred for the NMDA receptor blocker, since that route of administration provides greater ability to adjust and titer the dosage rate until the desired level is achieved and then sustained.

This invention grew out of the discovery and realization that a certain type of treatment involving two or more combined drugs, which was shown to provide lasting relief (and apparently even permanent relief, in most patients who have been treated so far) from a terrible and debilitating class of severe neuro-pathological problems that were totally incurable under the prior art, apparently created a permanent and stable improvement in those patients' nervous systems.

The type of relief that was demonstrated and proven, in the tests and treatments done to date, involved relief from intense, chronic, and previously incurable pain, in patients who suffer from the most difficult and intractable type of neuropathic pain syndrome known to medicine. As mentioned in the Background section, syndrome formerly was known as reflex sympathetic dsytrophy (RSD), indicating that it is a wasting disease, but that name was recently changed to a more neutral phrase, “complex regional pain syndrome, type 1” (CRPS-1). Since it was effectively incurable under the prior art, the Food and Drug Administration was willing to approve experimental treatments that could offer any relief to sufferers, despite the knowledge that such treatments posed serious risks to the patients being treated in those tests.

As described in the Background section, administration of clonidine, by one of the coiventors herein, to a test patient who was experiencing increasing blood pressure, led to the discovery that when clonidine is added to ketamine, the effectiveness of the pain relief that can be provided by both drugs, in combination, increases substantially. Additional research and discussions led to the realization that the risk of neurotoxic side effects that otherwise would be caused by sustained administration of ketamine also can be substantially reduced, by coadministering clonidine. Both of those benefits are highly important, and highly useful, and the results of the combined drug treatment on additional test patients have been truly extraordinary.

The realization that an extraordinarily effective treatment for a previously incurable neurological disorder had been achieved, on an apparently permanent basis, by using maximal tolerated dosages of a drug that specifically suppresses activity at the NMDA subclass of glutamate receptors in the mammalain nervous system, led to the subsequent realization that this treatment, using combined drugs, is creating and achieving a stable and long-term alteration and improvement, in a patient's neural homeostasis. Accordingly, this type of effect is referred to herein as an improved stable neural homeostasis, referred to herein by the acronym ISNH.

That realization (i.e., what is actually being created and achieved, by the combined drug treatment, qualifies as a stable and lasting alteration in a patient's neural homeostasis) led the inventors to the subsequent realization that this same treatment can also, in a comparable manner, be used to help patients who are suffering from other types of severe neurological disorders and impairments, which do not involve physical pain.

One such category of neurological disorders includes addictions, dependencies, and cravings that are seriously damaging people's lives. Such addictions, dependencies, and cravings often involve alcohol, nicotine, illegal drugs (such as marijuana, cocaine, heroin, amphetamines, LSD and other psychedelic and/or hallucinogenic drugs, Ecstasy, etc.), and various opiates, narcotics, and other pain-killing, sedative, anxiolytic and similar drugs. However, those are not the only types of cravings and addictions that are likely to be helped, in at least some patients, by the drug interventions disclosed herein. For example, in people who suffer from obesity or other problems that have reached a point of severely damaging or jeopardizing their life, health, and happiness, this type of drug treatment (especially if used in conjunction with skilled counseling and behavioral therapy) may be able to help at least some patients establish and achieve different and altered mental outlooks, attitudes, and thought patterns involving food, diet, and health, and it may help them control intense cravings for sweets, fatty foods, and other foods or treats that need to be avoided to bring that person's weight, health, and happiness under better control.

This treatment may also be able to provide major relief and possibly even cures, for at least some patients, for a variety of medical problems, other than neuropathic pain, that have important neurological components. In view of the mind-body connections that are being evaluated and explored by scientists and researchers (which involve spiritual factors only to a small extent, and which focus more heavily on known factors that can be studied, analyzed, and proven, such as the sympathetic and parasympathetic nervous systems, and how muscles and organs are “innervated” by multiple different types of neurons), this type of treatment, using a drug combination that can create lasting alterations in the NMDA receptor system and possibly other neuronal receptor systems as well, may be able to provide substantial relief for at least some people who suffer from disorders such as chronic fatigue syndrome, asthma, disorders that involve the immune system (such as severe allergies, hypersensitivity conditions, and multiple chemical sensitivity syndromes), lupus and other autoimmune disorders, and migraine, cluster, or other chronic or recurrent headaches. It may also be useful for treating chronic hyper-arousal, unwanted ideations or obsessive thoughts, and various types of sleep-related disorders, such as insomnia or narcolepsy.

This treatment also should be evaluated to determine whether it may be able to slow, prevent, or reduce the development, emergence, or severity of diabetes, in patients who are displaying pre-diabetic symptoms, or diabetic symptoms or problems that are early, mild, or incipient and have not yet progressed to full-blown diabetes.

Another category of problems that can be helped (in at least some patients) by this type of combined drug treatment involves people who suffer from one or more mental disorders. Candidate disorders that can be evaluated, to determine the extent to which patients in any such category will be helped and benefitted by such treatments, include obsessive-compulsive disorders, severe or chronic depression (either by itself, or as one component of a bipolar disorder), affective disorders, post-traumatic stress disorders, severe phobias, Tourette's syndrome, and possibly even some forms of schizophrenia, psychosis, dementia, or other mental illness. Such disorders also include sexual disorders that pose serious risks, either to a person suffering from a disorder, or to other people who might become victims of the person with the disorder. Such disorders include, for example, pedophilia, malevolent fetishes or aberrations that have passed beyond healthy limits and have reached a point of threatening a person's life or livelihood, and intensely strong and inappropriate fantasies or visions of rape, incest, or other actions that would severely damage or destroy people's lives or family, social, or work relationships, if actually committed.

Another category of problems involves people who act as through they are addicted to the excitement, peak energy levels, or other effects that come from taking extreme and/or unwarranted risks. Such people include compulsive gamblers, shoplifters who do not need what they steal, and people who repeatedly violate laws against driving while intoxicated or under the influence of alcohol or drugs. It may also include criminals, parolees, and others who engage in pursuits that needlessly jeopardize or damage themselves or innocent people, such as people who engage in embezzlement, fraud, swindling, or similar crimes, or who commit burglaries, rapes, armed robberies, or other crimes, because they are pathologically attracted or addicted to the thrill of feeling like they're living on the edge, and they're proving that they can do things normal people can't do.

It is not claimed or asserted by the Inventors herein that the combined drug treatments described herein can successfully and effectively treat any and all of these disorders, or that it can treat all patients who suffer from any one of these disorders. Instead, the claims and assertions herein can be summarized as follows:
1. Tests and treatments have shown that administration of a potent NMDA receptor blocker, at a maximum tolerated dosage, for several days continuously, can provide profound and lasting alterations, in the nervous systems of at least some patients who were suffering from terrible neurological disorders that could not be treated adequately by any other known treatment.
2. Despite their potentials for beneficial therapeutic use, potent NMDA receptor blockers clearly and undeniably cause serious and even severe damage, up to and including neuronal death and necrosis, in certain vulnerable regions of the brain, when administered to animals for sustained periods of time. Because various factors and correlations suggest that similar neurotoxic damage will also occur in the brains of humans who are treated with (or who illegally consume) such drugs, it is not safe or advisable to administer a potent NMDA antagonist, by itself, to any patient over a sustained span of time, using the dosages and time periods that will be required to create the type of stable and lasting alteration that needs to be achieved, in order to effectively treat a patient suffering from a severe neurological disorder.
3. In view of items 1 and 2, above, any physician who is treating a patient by sustained administration of a potent NMDA receptor blocker should also use a second drug that can reduce or provent the neurotoxic side effects and risks of the NMDA receptor blocker.
4. In addition to the safety factor described above, it has been discovered that at least one type of safener drug that has been tested to date (clonidine, which stimulates activity at the alpha-2 subclass of adrenergic receptors) can also increase the pain-relieving efficacy of a sustained ketamine treatment, in patients suffering from neuropathic pain. Because of the way these drugs function in a mammalian nervous system, and because of the adaptive, responsive, homeostatic nature of a mammalian nervous system, it is believed and anticipated that clonidine (and possibly other alpha-2 adrenergic agonists as well) will also be able to increase, in at least some patients, the efficacy of an NMDA receptor blocker (such as ketamine) in establishing stable and long-lasting alterations and improvements in the neural homeostasis of patients who suffer from severe neurologic disorders that are not related to physical pain, and that instead have primarily mental, emotional, or behavioral manifestations.
5. As with any other type of medical treatment, different patients who have differing histories, conditions, physiologies, and genomes are likely to respond in somewhat different ways, which likely can be plotted on conventional “bell curve” distributions for any particular group of patients and/or disorders. The Inventors herein do not claim or assert that all patients will respond in desirable or predictable manners. Nevertheless, the Inventors herein assert that the combined drug treatments disclosed herein will be able to offer substantial and beneficial relief, and improvement, to at least some of the patients who are tested and/or treated by these methods.
6. Because of the nature of these treatments, which use potent drugs to create lasting neurological alterations (which may well include mental, psychological, emotional, behavioral, or other alterations) in treated patients, these treatments should be tested, evaluated, and approached with caution and restraint. Careful and extensive screening and pre-testing of candidate patients should be carried out, prior to such treatment being seriously considered for any candidate. In addition, any treatment should be preceded, accompanied, and followed by skilled professional counseling, to maximize the likelihood of a desired and/or optimal outcome for that individual patient.
   Additional Agents; Magnesium Salt, CYP3A4 Enzyme Inhibitor

Intravenous administration of a water-soluble magnesium salt (such as magnesium sulfate, which is readily available in injectable formulations) is generally preferred and should be considered by any treating physician, since it has been shown to provide additive or synergistic beneficial effects in some of the treatments carried out to date on patients suffering from neuropathic pain. Magnesium is believed to have moderate and mild NMDA receptor blocking activity, and magnesium administration appears to supplement and enhance the action of ketamine as a primary NMDA receptor blocker. Based on general principles of chemical and biological equilibrium, the assumption and belief is that by providing two different agents that can work simultaneously and in harmony on a single receptor system, a drug treatment using lower dosages of those two compatible drugs may be able to spread out and exert its effects in a more balanced, even, well-distributed manner, compared to a treatment using a higher dosage of a single drug.

Similarly, any treating physician who is carrying out a ketamine-plus-safener treatment as disclosed herein should also be aware that many but not all patients rapidly metabolize ketamine, in a way that removes the methyl group from the aminomethyl side chain on ketamine, converting it into a less active metabolite called norketamine. This conversion is carried out mainly by an enzyme that is usually referred to as CYP3A4 (Hijazi et al 2002; the “CYP” prefix indicates that this enzyme belongs within a family of enzymes referred to as cytochrome P450 enzymes). This enzyme is active in both intestinal tissue (which can reduce the amount of ketamine that reaches the bloodstream) and in the liver (which will remove ketamine from circulating blood).

Several agents are known that can inhibit the CYP3A4 enzyme, thereby helping to sustain elevated ketamine levels in circulating blood during a treatment. One effective and inexpensive CYP3A4 inhibitor is contained in grapefruit juice (e.g., Veronese et al 2003). Therefore, unless a patient has been screened in advance to ensure that his or her levels of ketamine in blood will remain high without additional treatment by a CYP3A4 inhibitor, any treating physician should seriously consider having any patient drink a moderately large glass of grapefruit juice, twice a day, starting a day or two before the treatment begins, and continuing throughout the course of the treatment. This is a low-cost and convenient way to help ensure that ketamine levels in the blood will remain at desirably high levels, throughout the treatment. Alternately, if the patient has an aversion to grapefruit juice, other CYP3A4 inhibitors that can be taken in capsule form are known, such as ketoconazole. If ketoconazole is used, the “levorotatory” stereoisomer is more potent and preferable (Dilmaghanian et al 2004).

Anti-cholinergic drugs also merit particular attention, because of how they work in general, and because they may be useful, directly as safener agents. As mentioned in the Background section, glutamate (which works through NMDA, kainate, and AMPA receptors) and acetyl-choline (which works through two families of receptors, known as nicotinic and muscarinic receptors) are the two most important types of excitatory neurotransmitters, in any higher animal (most other neurotransmitters, such as dopamine, serotonin, and GABA, are inhibitory rather than excitatory). Therefore, if a mild level of suppression of the acetyl-choline system is created in a patient undergoing treatment as described herein, the combined suppression of excitatory activity at both NMDA and acetylcholine receptors may have an even greater, more potent, and more rapid effect in enabling a patient's nervous system to move toward a healthy, pain-free, undistressed “set-point” of the type that was enjoyed before a neurological disorder began.

This possibility (i.e., adding a mild anti-cholinergic drug, both as a safener agent, and as a potentially helpful additive or synergistic active agent, to an NMDA blockade treatment) merits evaluation, fairly early in the process of evaluating these types of treatments for various specific types of neurologic disorders. Such additive treatments can be provided and tested, by using any of numerous known anti-cholinergic agents, including scopolamine or atropine, or possibly procyclidine or ethopropazine, which also have mild levels of NMDA receptor blocking activity. If it is shown that supplementary anti-cholinergic treatment provides improved results in treating any one or more particular types of neurological disorders, it can thereafter be incorporated into any such treatments, as an enhancement, at the discretion of the treating physician.

It also should be noted that clonidine does not have especially high selectivity for the alpha-2 subclass of adrenergic receptors, and published reports have stated that certain other a2 agonists (including dexmedetomidine, guanfacine, and azepexole, and possibly lofexidine) have greater selectivity for the alpha-2 subclass in particular. Accordingly, any of those agents (or any other known or hereafter-discovered alpha-2 adrenergic agonist) can be tested in the types of treatments disclosed herein, to compare their efficacy to clonidine when used in such treatments. Various other known alpha-2 adrenergic agonists include iodoclonidine, guanabenz, xylazine, medetomidine, tizanidine, rilmenidine, alpha-methyldopa, and alpha-methylnoradrenaline.

If desired, any other active agent can also be administered to a patient, during a treatment as described herein, if the treating physician who has evaluated the patient's condition and history believes that such agent may help the treatment, or at least is not likely to interfere with it. Such agents might include, for example, prescription or non-prescription medications that a patient was taking before the treatment began. During any such treatment, consideration should be given to whether it would be appropriate to begin tapering off the dosages of any such additional medication, during the course of the treatment.

Similarly, various types of anti-oxidants, anti-apoptotic agents, vitamins, and other potentially beneficial agents can also be evaluated for use in such treatments. Examples of such agents that are regarded as meriting serious evaluation include vitamins C and E, the carotenoids lycopene and zeaxanthin, selenium and possibly other minerals (possibly including zinc, although zinc and copper recently have been implicated in the beta-amyloid plaques of Alzheimer's disease, and in certain forms of neurotoxicity following a stroke or other crisis), and various types of anti-apoptotic mitochondrial stabilizers.

### EXAMPLES

The examples set forth below are based on treatments using sustained-dosage ketamine, for severe and intractable neuropathic pain. Most of these patients suffered from reflex sympathetic dystrophy (also called complex regional pain syndrome, type 1), which is one of the most difficult and intractable forms of chronic neuropathic pain known to medicine. Therefore, the results reported below provide confirmation that patients who were successfully treated did indeed undergo a substantial and stable improvement in their neural condition, which lasted long after the treatments ended.

Accordingly, these examples are offered to set forth detailed dosage regimens of the type that have been developed and tested to date, and that have been shown to create and achieve an “improved stable neural homeostasis”, as that term is used herien, in patients who were suffering from severe neurologic disorders.

### Example 1

### Female Patient with CRPS-1 (RSD)

A female in her 40s had suffered for roughly 9 years from intractable chronic pain, diagnosed as CRPS-1 and/or RSD, in her right leg and foot. This pain apparently was related to circulatory problems in her right leg. Her medical status prior to the ketamine treatment, the details of her treatment, and her pain condition after the treatment, have been described in detail in Harbut and Correll 2002 (written by two of the inventors herein, and not conceded to be prior art). The contents of that article are incorporated herein by reference.

This patient was pain-free for roughly 18 months. At that point in time, a clot formed in her right leg, provoking circulatory problems. This triggered the return of a pain condition, but she reported that it was not as bad as what she had suffered prior to the ketamine treatment.

### Example 2

### Female Patient with CRPS-1 (RSD)

A female in her 20's had a two-year history of severe chronic pain in her left fingers, hand, arm, and shoulder. She was taking sustained-release morphine, at roughly 100 mg/day.

She was treated with 2 mg of MgSO4 (infused in 5% dextrose in water, or D5W) and 0.1 mg clonidine (oral tablet) prior to ketamine. Throughout the treatment, she continued to receive 0.1 mg of clonidine every 12 hours. Ketamine was initially infused at 10 mg/hour, and was gradually increased to 20 mg/hr, until it began to show CNS effects, such as slurring of speech (those side effects resolved within about 24 hours). A second magnesium infusion was performed at 24 hr.

She reported essentially complete relief from her pain after about 2-3 days of ketamine infusion. The last time the treating physician communicated her, about 12 months after her treatment, she was still pain-free.

### Example 3

### Male Patient with Herpetic Shingles

A male in his early 40's had suffered for about 1.5 years from intractable post-herpetic trigeminal neuralgia (commonly known as shingles). He was maintained on ketamine infusion for only 48 hours, and the clonidine portion of his treatment did not start before the ketamine infusion began; instead, it was commenced during the first day of infusion, to help control his blood pressure.

This patient also had a marginally low serum magnesium, so he was given 2 grams of MgSO4 prior to the ketamine infusion, and another 2 g later in the ketamine infusion. His response provided the first solid indication and confirmation that magnesium could have a synergistic pain-reducing effect, if coadministered along with ketamine.

The last contact the treating physician had with this patient was about 12 months after his treatment. His pain was still largely abated, with periodic flareups that he described as minor, and nothing like the pain he had previously suffered.

Thus, there has been shown and described a new and useful means for using sustained administration of an NMDA receptor blocker drug, such as ketamine, for treating a variety of neurological disorders, in ways that will give a patient's damaged or stressed nervous system a period of quiet rest, so that it can move back in the direction of a healthy and desirable neural homeostasis. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Berman, R. M., et al, “Antidepressant effects of ketamine in depressed patients,” Biol Psychiatry 47: 351-4 (2000)
Corso, T. D., et al, “Multifocal brain damage induced by phencyclidine is augmented by pilocarpine,” Brain Research 752: 1-14 (1997)
Dilmaghanian, S., et al, “Enantioselectivity of inhibition of cytochrome P450 3A4 (CYP3A4) by ketoconazole,” Chirality 16: 79-85 (2004)
Fitzgibbon, E. J., et al, “Low dose ketamine as an analgesic adjuvant in difficult pain syndromes: a strategy for conversion from parenteral to oral ketamine,” J Pain Symptom Management 23: 165-70 (2002)
Fix, A. S., et al, “Pathmorphologic effects of NMDA antagonists in the rat posterior-cingulate retrosplenial cerebral cortex: A review,” Drug Development Research 24: 147-152 (1994)
Fix, A. S., et al, “Neuronal vacuolization and necrosis induced by the noncompetitive NMDA antagonist MK-801: A light and electron microscopic evaluation of the rat retrosplenial cortex,” Exp. Neurology 123: 204-215 (1993)
Harbut, R. E., and Correll, G. E., “Successful treatment of a nine-year case of CRDS Type 1 . . . ” Pain Medicine 3: 147-155 (2002)
Hijazi, Y., et al, “Contribution of CYP3A4, CYP2B6, and CYP2C9 isoforms to N-demethylation of ketamine in human liver microsomes,” Drug Metab Dispos. 30: 853-8 (2002)
Kannan, T. R., et al, “Oral ketamine as an adjuvant to oral morphine for neuropathic pain in cancer patients,” J Pain Symptom Management 23: 60-5 (2002)
Klepstad, P., et al, “Long-term treatment with ketamine in a 12-year-old girl with severe neuropathic pain caused by a cervical spinal tumor,” J Pediatr Hematol Oncol 23: 616-9 (2001)
Mitchell, A. C., “An unusual case of chronic neuropathic pain responds to an optimum frequency of intravenous ketamine infusions,” J Pain Symptom Manage 21: 443-6 (2001)
Olney, J. W., et al, “NMDA antagonist neurotoxicity: Mechanism and prevention,” Science 254: 1515-1518 (1991)
Rabben, T. et al, “Interindividual differences in the analgesic response to ketamine in chronic orofacial pain,” Eur J Pain 5: 233-40 (2001)
Sanacora, G., et al, “Clinical studies implementing glutamate neurotransmission in mood disorders,” Ann N Y Acad. Sci. 1003: 292-308 (2003)
Veronese, M. L., et al, “Exposure-dependent inhibition of intestinal and hepatic CYP3A4 in vivo by grapefruit juice,” J Clin Pharmacol. 43: 831-9 (2003)

1. A method for treating a neurologic disorder other than chronic or neuropathic pain, comprising the step of coadministering to a patient in need of such treatment:
a. at least one NMDA receptor antagonist drug, at a dosage-time combination that has been shown in human trials to be effective in creating stable and long-term neurological alterations in treated patients; and,
b. at least one safener drug that has been shown, using in vivo animal tests, to reduce neurotoxic side effects caused by MK-801 when administered without an accompanying safener drug.

2. The method of claim 1, wherein the dosage of the NMDA receptor antagonist drug is titered for the patient being treated, to a level that causes slurring of speech in that patient but does not render the patient unconscious.

3. The method of claim 1, wherein the NMDA receptor antagonist drug comprises ketamine, and wherein the ketamine is administered to the patient by intravenous infusion over a continuous period of at least 36 hours, at a dosage sufficient to establish and sustain a blood concentration of at least about 200 nanograms of ketamine per milliliter of blood plasma for a period of at least 24 hours.

4. The method of claim 3, wherein the ketamine is administered to the patient at a dosage sufficient to establish and sustain a blood concentration of at least about 250 nanograms of ketamine per milliliter of blood plasma for a period of at least 40 hours.

5. The method of claim 1, wherein the safener drug comprises an alpha-2 adrenergic agonist drug.

6. The method of claim 5, wherein the alpha-2 adrenergic agonist drug is selected from the group consisting of clonidine, iodoclonidine, guanabenz, xylazine, medetomidine, tizanidine, rilmenidine, alpha-methyldopa, alpha-methylnoradrenaline, guanfacine, dexmedetomidine, azepexole, and lofexidine.

7. The method of claim 1, wherein a water-soluble magnesium salt is also coadministered to the patient.

8. The method of claim 1, where an enzyme inhibitor that slows metabolic degradation of ketamine in circulating blood is also coadministered to the patient.

9. The method of claim 1, where the enzyme inhibitor inhibits the CYP3A4 cytochrome P450 enzyme.

10. A method for treating a neurologic disorder other than chronic or neuropathic pain, comprising the step of administering to a patient in need of such treatment at least one NMDA receptor antagonist drug that is known to have an inherent safening activity due to activity at a second type of neuronal receptor, at a dosage that causes slurring of speech without causing hallucinations or loss of consciousness, and at a dosage-time combination that has extends for at least three days in succession, and that has been shown in human trials to be effective in creating stable and long-term neurological alterations in treated patients.

11. The method of claim 10, wherein the NMDA receptor antagonist drug that is known to have an inherent safening activity due to activity at a second type of neuronal receptor is selected from the group consisting of ibogaine, eliprodil, and analogs and derivatives thereof that are effective as NMDA receptor antagonist drugs with inherent safening activity due to activity at a second type of neuronal receptor.
